(19) United States
(12) Patent Application Publication
(10) Pub. No.: US 2023/0136342 A1
(43) Pub. Date: May 4, 2023

(54) SYSTEMS AND METHODS FOR DETECTING CELL-ASSOCIATED BARCODES FROM SINGLE-CELL PARTITIONS

(71) Applicant: 10x Genomics, Inc., Pleasanton, CA (US)

(72) Inventors: Daniel P. Riordan, Hillsborough, NC (US); Maria Alexis, Oakland, CA (US); Joseph Glenn Arthur, Oakland, CA (US); Andrew John Hill, Oakland, CA (US)

(21) Appl. No.: 18/090,814

(22) Filed: Dec. 29, 2022

Related U.S. Application Data
(63) Continuation of application No. PCT/US2021/040165, filed on Jul. 1, 2021.
(60) Provisional application No. 63/047,896, filed on Jul. 2, 2020.

(51) Int. Cl. G16B 25/10; G16B 40/20; G16B 40/30

(52) U.S. Cl. CPC G16B 25/10; G16B 40/20; G16B 40/30

(57) ABSTRACT

Method for identifying a subset of cells from a dataset for targeted gene expression analysis, comprising: defining an expected total recovered cell count for a barcode dataset; determining a region of interest for the total recovered cell count by calculating a lower bound for the region; and (b) calculating an upper bound for the region of interest by including an additional subset of barcodes having molecule counts below the lower bound; fitting the barcode dataset to a model; wherein the model groups barcodes as a function of molecule count; identifying a cut-off value on the model, wherein all barcodes having a molecule count above the cut-off value are called as cells; and identifying a targeted subset of cells from the called cells for conducting targeted gene expression, wherein a cell qualifies as part of the targeted subset if the cell has a molecule count above a target threshold.

### APPLICATIONS FOR CLAIM OF PRIORITY

The present application is a continuation under 35 U.S.C. 111(a) of PCT/US2021/040165, filed on Jul. 1, 2021, entitled “SYSTEMS AND METHODS FOR DETECTING CELL-ASSOCIATED BARCODES FROM SINGLE-CELL PARTITIONS,” which claims priority to U.S. Provisional Patent Application No. 63/047,896, filed on Jul. 2, 2020, entitled “SYSTEMS AND METHODS FOR DETECTING CELL-ASSOCIATED BARCODES FROM SINGLE-CELL PARTITIONS,” which applications are herein incorporated by reference in their entirety for all purposes.

### FIELD

The embodiments provided herein are generally related to systems and methods for analysis of genomic nucleic acids or targeted gene expression and classification of those features. Included among embodiments provided herein are systems and methods relating to accurate detection of cell-associated barcodes.

### BACKGROUND

Accurate detection of cell-associated barcodes such as, for example, barcodes from partitions containing one or more cells, is a primary step in the analysis of single-cell molecular datasets from barcoded partitions. Correct cell-calling remains an important challenge for the successful analysis of unbiased genome-wide single-cell molecular datasets (datasets comprising, for example, genomic or targeted expression data). However, this problem becomes even more challenging when addressing targeted single-cell datasets, due to the additional complications involved with performing targeted enrichment steps to selectively recover a subset of targeted molecules from the overall population, and their associated impact on features of the resulting targeted datasets.

As such, there is a need for better classification of cell-associated barcodes in a wide variety of single-cell datasets. Moreover, there is a need to identify solutions that more effectively classify cell-associated barcodes with targeted single-cell datasets.

### SUMMARY

In accordance with various embodiments, a method is provided for identifying a subset of cells from a dataset for targeted gene expression analysis, the method comprising defining an expected total recovered cell count for a barcode dataset, wherein the barcodes in the dataset are sorted based on count; determining a region of interest for the total recovered cell count by calculating a lower bound for the region of interest using a percentile of the sorted barcodes, wherein all barcodes having a molecule count above the lower bound are included for further analysis; calculating an upper bound for the region of interest by including an additional subset of barcodes having molecule counts below the lower bound, wherein each barcode in the subset that has a molecule count below a count threshold is excluded from further analysis; fitting the barcode dataset to a model, wherein the model groups barcodes as a function of molecule count; identifying a cut-off value on the model, wherein all barcodes having a molecule count above the cut-off value are called as cells; and identifying a targeted subset of cells from the called cells for conducting targeted gene expression, wherein a cell qualifies as part of the targeted subset if the cell has a molecule count above a target threshold.

In accordance with various embodiments, a non-transitory computer-readable medium storing computer instructions is provided for identifying a subset of cells from a dataset for targeted gene expression analysis, the method comprising defining, by one or more processors, an expected total recovered cell count for a barcode dataset, wherein the barcodes in the dataset are sorted based on count; determining, by one or more processors, a region of interest for the total recovered cell count by calculating a lower bound for the region of interest using a percentile of the sorted barcodes, wherein all barcodes having a molecule count above the lower bound are included for further analysis; calculating an upper bound for the region of interest by including an additional subset of barcodes below the lower bound, wherein each barcode in the subset that has a molecule count below a count threshold is excluded from further analysis; fitting, by one or more processors, the barcode dataset to a model, wherein the model groups barcodes as a function of molecule count; identifying, by one or more processors, a cut-off value on the model, wherein all barcodes having a molecule count above the cut-off value are called as cells; and identifying, by one or more processors, a targeted subset of cells from the called cells for conducting targeted gene expression, wherein a cell qualifies as part of the targeted subset if the cell has a molecule count above a target threshold.

In accordance with various embodiments, a system is provided for identifying a subset of cells from a genomic sequence dataset for targeted gene expression analysis, comprising: a data store configured to store the genomic sequence dataset comprising a plurality of fragment sequence reads, each with an associated barcode sequence and a unique identifier sequence; and a computing device communicatively connected to the data store, comprising a unique molecule filtering engine configured to define an expected total recovered cell count for a barcode dataset, wherein the barcodes in the dataset are sorted based on count; determine a region of interest for the total recovered cell count by (a) calculating a lower bound for the region of interest using a percentile of the sorted barcodes, wherein all barcodes having a molecule count above the lower bound are included for further analysis; and (b) calculating an upper bound for the region of interest by including an additional subset of barcodes below the lower bound, wherein each barcode in the subset that has a molecule count below a count threshold is excluded from further analysis; fit the barcode dataset to a model, wherein the model groups barcodes as a function of molecule count; identify a cut-off value on the model, wherein all barcodes having a molecule count above the cut-off value are called as cells; and identify a targeted subset of cells from the called cells for conducting targeted gene expression, wherein a cell qualifies as part of the targeted subset if the cell has a molecule count above a target threshold.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIGS. 1A and 1B are schematic illustrations of non-limiting examples of the sequencing workflow for using single cell targeted gene expression sequencing analysis to generate sequencing data for analyzing the expression profile of targeted genes of interest, in accordance with various embodiments.

FIG. 2 is an exemplary flowchart showing a process flow for conducting for gene expression analysis, in accordance with various embodiments.

FIG. 3 is an exemplary flowchart showing a process flow for conducting for targeted gene expression analysis, in accordance with various embodiments.

FIG. 4 is an exemplary flowchart showing a process flow for conducting for targeted gene expression analysis, in accordance with various embodiments.

FIG. 5 is an exemplary flowchart showing a process flow for identifying a subset of cells from a genomic sequence dataset for targeted gene expression analysis, in accordance with various embodiments

FIG. 6 is a schematic diagram of a system for identifying a subset of cells from a genomic sequence dataset for targeted gene expression analysis, in accordance with various embodiments.

FIG. 7 is a chart depicting a log-transformed barcode-rank plot, in accordance with various embodiments.

FIG. 8 is a chart depicting a log-transformed barcode-rank plot, in accordance with various embodiments.

FIG. 9 is a chart depicting the number of identified cell-associated barcodes from a particular single-cell sample versus an associated sample identifier index, in accordance with various embodiments.

FIG. 10 is a chart depicting the number of identified cell-associated barcodes from a particular single-cell sample versus an associated sample identifier index, in accordance with various embodiments.

FIG. 11 is a chart depicting the number of identified cell-associated barcodes from a particular single-cell sample versus an associated sample identifier index, in accordance with various embodiments

FIG. 12 is a chart depicting the number of identified cell-associated barcodes from a particular single-cell sample versus an associated sample identifier index, in accordance with various embodiments

FIGS. 13A-D show bar graphs depicting the mean (left panels, 13A and 13C) and median (right panels, 13B and 13D) deviation between the number of barcodes called as cells across the set of targeted gene expression samples analyzed relative to the number of barcodes called as cells in each corresponding input WTA sample, in accordance with various embodiments.

FIG. 14 is a block diagram illustrating a computer system for use in performing methods provided herein, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

The above-identified figures are provided by way of representation and not limitation. The figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

Provided herein are methods and systems for identifying a subset of cells from a genomic sequence dataset for targeted gene expression analysis. It should be appreciated, however, that although the systems and methods disclosed herein refer to their application in targeted gene expression analysis workflows, they are equally applicable to other analogous fields.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any use of subheadings herein is for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

All publications mentioned herein are incorporated herein by reference for the purpose of describing and disclosing devices, compositions, formulations and methodologies which are described in the publication and which might be used in connection with the present disclosure.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, un-recited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. Standard molecular biological techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and standard techniques described herein are those well-known and commonly used in the art.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

DNA (deoxyribonucleic acid) is a chain of nucleotides containing 4 types of nucleotides; A (adenine), T (thymine), C (cytosine), and G (guanine), and RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

A term “genome”, as used herein, refers to the genetic material of a cell or organism, including animals, such as mammals, e.g., humans and comprises nucleic acids, such as DNA. In humans, total DNA includes, for example, genes, noncoding DNA and mitochondrial DNA. The human genome typically contains 23 pairs of linear chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

As used herein, the phrase “genomic feature” refers to a defined or specified genome element or region. In some instances, the genome element or region can have some annotated structure and/or function (e.g., a chromosome, a gene, protein coding sequence, mRNA, tRNA, rRNA, repeat sequence, inverted repeat, miRNA, siRNA, etc.) or be a genetic/genomic variant (e.g., single nucleotide polymorphism/variant, insertion/deletion sequence, copy number variation, inversion, etc.) which denotes one or more nucleotides, genome regions, genes or a grouping of genome regions or genes (in DNA or RNA) that have undergone changes as referenced against a particular species or sub-populations within a particular species due to, for example, mutations, recombination/crossover or genetic drift.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM), Ion Torrent, and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing process performed to determine some information relating to at least one biomolecule (e.g., nucleic acid molecule). The term can also refer to a flowcell containing data from one sequencing instrument run. The sequencing data can be further addressed by lane and by one or more sample indices.

The term “read” with reference to nucleic acid sequencing refers to the sequence of nucleotides determined for a nucleic acid fragment that has been subjected to sequencing, such as, for example, NGS. Reads can be any a sequence of any number of nucleotides which defines the read length.

The phrase “sequencing coverage” or “sequence coverage,” used interchangeably herein, generally refers to the relation between sequence reads and a reference, such as, for example, the whole genome of cells or organisms, one locus in a genome or one nucleotide position in the genome. Coverage can be described in several forms (see, e.g., Sims et al. (2014) Nature Reviews Genetics 15:121-132). For example, coverage can refer to how much of the genome is being sequenced at the base pair level and can be calculated as NL/G in which N is the number of reads, L is the average read length, and G is the length, or number of bases, of the genome (the reference). For example, if a reference genome is 1000 Mbp and 100 million reads of an average length of 100 bp are sequenced, the redundancy of coverage would be 10×. Such coverage can be expressed as a “fold” such as 1×, 2×, 3×, etc. (or 1, 2, 3, etc. times coverage). Coverage can also refer to the redundancy of sequencing relative to a reference nucleic acid to describe how often a reference sequence is covered by reads, e.g., the number of times a single base at any given locus is read during sequencing. Thus, there may be some bases which are not covered and have a depth of 0 and some bases that are covered and have a depth of anywhere between, for example, 1 and 50. Redundancy of coverage provides an indication of the reliability of the sequence data and is also referred to as coverage depth. Redundancy of coverage can be described with respect to “raw” reads that have not been aligned to a reference or to aligned (e.g., mapped) reads. Coverage can also be considered in terms of the percentage of a reference (e.g., a genome) covered by reads. For example, if a reference genome is 10 Mbp and the sequence read data maps to 8 Mbp of the reference, the percentage of coverage would be 80%. Sequence coverage can also be described in terms of breadth of coverage which refers to the percentage of bases of a reference that are sequenced a given number of times at a certain depth.

In some cases, a reference may be defined as a particular set of identified molecules or transcripts which are derived from a genome or transcriptome (e.g. a region of interest). Such a reference is particularly applicable to single-cell datasets. For example, in the calculation NL/G, N is the number of reads, L is the average read length, and G is the length, or number of bases, of the identified molecules or transcripts (the reference).

In general, the methods and systems described herein accomplish targeted genomic sequencing (including targeted gene expression sequencing) by providing for the determination of the sequence of long individual nucleic acid molecules and/or the identification of direct molecular linkage as between two sequence segments separated by long stretches of sequence, which permit the identification and use of long range sequence information, but this sequencing information is obtained using methods that have the advantages of the extremely low sequencing error rates and high throughput of short read sequencing technologies. The methods and systems described herein segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long range molecular context is not only preserved through a sequencing process, but is also preserved through the targeted enrichment process used in targeted sequencing approaches described herein, where no other sequencing approach has shown this ability.

In general, sequence information from smaller fragments will retain the original long range molecular sequence context through the use of a tagging procedure, including the addition of barcodes as described herein and known in the art. In specific examples, fragments originating from the same original longer individual nucleic acid molecule will be tagged with a common barcode, such that any later sequence reads from those fragments can be attributed to that originating longer individual nucleic acid molecule. Such barcodes can be added using any method known in the art, including addition of barcode sequences during amplification methods that amplify segments of the individual nucleic acid molecules as well as insertion of barcodes into the original individual nucleic acid molecules using transposons, including methods such as those described in Amini et al., Nature Genetics 46: 1343-1349 (2014) (advance online publication on Oct. 29, 2014), which is hereby incorporated by reference in its entirety for all purposes and in particular for all teachings related to adding adaptor and other oligonucleotides using transposons. Once nucleic acids have been tagged using such methods, the resultant tagged fragments can be enriched using methods described herein such that the population of fragments represents targeted regions of the genome. As such, sequence reads from that population allows for targeted sequencing of select regions of the genome, and those sequence reads can also be attributed to the originating nucleic acid molecules, thus preserving the original long range molecular sequence context. The sequence reads can be obtained using any sequencing methods and platforms known in the art and described herein.

In addition to providing the ability to obtain sequence information from targeted regions of the genome, the methods and systems described herein can also provide other characterizations of genomic material, including without limitation haplotype phasing, identification of structural variations, and identifying copy number variations, as described in co-pending applications U.S. Ser. Nos. 14/752,589 and 14/752,602, both filed on Jun. 26, 2015), which are herein incorporated by reference in their entirety for all purposes and in particular for all written description, figures and working examples directed to characterization of genomic material.

In addition to providing the ability to obtain sequence information relating to the other characterizations above, the methods and systems described herein can also enable characterization of RNA-related variations present in expressed transcripts, including, but not limited to isoforms, splicing variants, and fusion information, as well as information relating to SNPs, SNVs, deletions, insertions, rearrangements, or polymorphisms. The methods and systems described herein allow for characterization of these variations on an individual basis, all at once, or in any desired combination.

Methods of processing and sequencing nucleic acids in accordance with the methods and systems described in the present application are also described in further detail in U.S. Ser. Nos. 14/316,383; 14/316,398; 14/316,416; 14/316,431; 14/316,447; and 14/316,463 which are herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing reads.

As used herein, the term “cell barcode” refers to any barcodes that have been determined to be associated with a cell, as determined by a “cell calling” step within various embodiments of the disclosure.

As used herein, the term “Gel bead-in-EMulsion” or “GEM” refers to a droplet containing some sample volume and a barcoded gel bead, forming an isolated reaction volume. When referring to the subset of the sample contained in the droplet, the term “partition” may also be used. In various embodiments within the disclosure, the term barcode can refer to a GEM containing a gel bead that carries many DNA oligonucleotides with the same barcode, whereas different GEMs have different barcodes.

As used herein, the term “GEM well” or “GEM group” refers to a set of partitioned cells (i.e., Gel beads-in-Emulsion or GEMs) from a single 10× Chromium™ Chip channel. One or more sequencing libraries can be derived from a GEM well.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches. In various embodiments within the disclosure, the term adapter can refer to customized strands of nucleic acid base pairs created to bind with specific nucleic acid sequences, e.g., sequences of DNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “macromolecule” or “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “sample,” as used herein, generally refers to a “biological sample” of a subject. The sample may be obtained from a tissue of a subject. The sample may be a cell sample. A cell may be a live cell. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. In some embodiments, the term “sample” can refer to a cell or nuclei suspension extracted from a single biological source (blood, tissue, etc.).

The sample may comprise any number of macromolecules, for example, cellular macromolecules. The sample maybe or may include one or more constituents of a cell, but may not include other constituents of the cell. An example of such cellular constituents is a nucleus or an organelle. The sample may be or may include DNA, RNA, organelles, proteins, or any combination thereof. The sample may be or include a chromosome or other portion of a genome. The sample may be or may include a bead (e.g., a gel bead) comprising a cell or one or more constituents from a cell, such as DNA, RNA, a cell nucleus, organelles, proteins, or any combination thereof, from the cell. The sample may be or may include a matrix (e.g., a gel or polymer matrix) comprising a cell or one or more constituents from a cell, such as DNA, RNA, nucleus, organelles, proteins, or any combination thereof, from the cell.

As used herein, the term “PCR duplicates” refers to duplicates created during PCR amplification. During PCR amplification of the fragments, each unique fragment that is created may result in multiple read-pairs sequenced with near identical barcodes and sequence data. These duplicate reads are identified computationally, and collapsed into a single fragment record for downstream analysis.

### Single Cell Sequencing and Data Analysis Workflows

Single Cell Targeted Gene Expression Sequencing Workflow

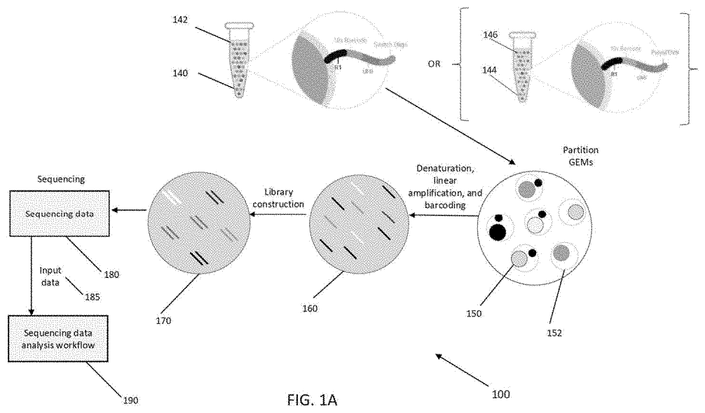

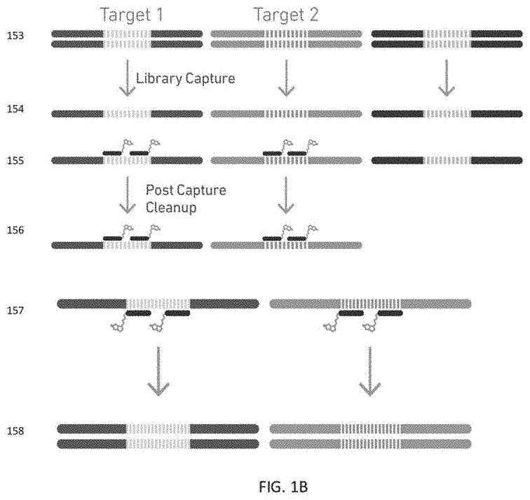

In accordance with various embodiments, a general schematic workflow is provided in FIG. 1 to illustrate a non-limiting example process for using single cell sequencing technology to generate sequencing data. Such sequencing data can be used for targeted gene expression analysis in accordance with various embodiments. The workflow can include various combinations of features, whether it be more or less features than that illustrated in FIG. 1. As such, FIG. 1 simply illustrates one example of a possible workflow.

GEM Generation

The workflow 100 provided in FIG. 1A begins with Gel beads-in-EMulsion (GEMs) generation. The bulk cell suspension containing the cells is mixed with a gel beads solution 140 or 144 containing a plurality of individually barcoded gel beads 142 or 146. In various embodiments, this step results in partitioning the cells into a plurality of individual GEMs 150, each including a single cell, and a barcoded gel bead 142 or 146. This step also results in a plurality of GEMs 152, each containing a barcoded gel bead 142 or 146 but no nuclei. Detail related to GEM generation, in accordance with various embodiments disclosed herein, is provided below. Further details can be found in U.S. Pat. Nos. 10,343,166 and 10,583,440, US Published Application Nos. US20180179590A1, US20190367969A1, US20200002763A1, and US20200002764A1, and Published International PCT Application No. WO 2019/040637, each of which is incorporated herein by reference in its entirety.

In various embodiments, GEMs can be generated by combining barcoded gel beads, individual cells, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 142 or 146 of the various embodiments herein may include a gel bead attached to oligonucleotides containing (i) an Illumina® P5 sequence (adapter sequence), (ii) a 16 nucleotide (nt) 10× Barcode, and (iii) a Read 1 (Read 1N) sequencing primer sequence. It is understood that other adapter, barcode, and sequencing primer sequences can be contemplated within the various embodiments herein.

In various embodiments, GEMS are generated by partitioning the cells using a microfluidic chip. To achieve single cell resolution per GEM, the cells can be delivered at a limiting dilution, such that the majority (e.g., ^{˜}90-99%) of the generated GEMs do not contain any cells, while the remainder of the generated GEMs largely contain a single cell.

Barcoding RNA Molecules or Fragments

The workflow 100 provided in FIG. 1A further includes lysing the cells and barcoding the RNA molecules or fragments for producing a plurality of uniquely barcoded single-stranded nucleic acid molecules or fragments. Upon generation of the GEMs 150, the gel beads 142 or 146 can be dissolved releasing the various oligonucleotides of the embodiments described above, which are then mixed with the RNA molecules or fragments resulting in a plurality of uniquely barcoded single-stranded nucleic acid molecules or fragments 160 following a nucleic acid extension reaction, e.g., reverse transcription of mRNA to cDNA, within the GEMs 150. Detail related to generation of the plurality of uniquely barcoded single-stranded nucleic acid molecules or fragments 160, in accordance with various embodiments disclosed herein, is provided below.

In various embodiments, upon generation of the GEMs 150, the gel beads 142 or 146 can be dissolved, and oligonucleotides of the various embodiments disclosed herein, containing a capture sequence, e.g., a poly(dT) sequence or a template switch oligonucleotide (TSO) sequence, a unique molecular identifier (UMI), a unique 10× Barcode, and a Read 1 sequencing primer sequence can be released and mixed with the RNA molecules or fragments and other reagents or a combination of biochemical reagents (e.g., a master mix necessary for the nucleic acid extension process). Denaturation and a nucleic acid extension reaction, e.g., reverse transcription, within the GEMs can then be performed to produce a plurality of uniquely barcoded single-stranded nucleic acid molecules or fragments 160. In various embodiments herein, the plurality of uniquely barcoded single-stranded nucleic acid molecules or fragments 160 can be 10× barcoded single-stranded nucleic acid molecules or fragments. In one non-limiting example of the various embodiments herein, a pool of 750,000, 10× barcodes are utilized to uniquely index and barcode nucleic acid molecules derived from the RNA molecules or fragments of each individual cell.

Accordingly, the in-GEM barcoded nucleic acid products of the various embodiments herein can include a plurality of 10× barcoded single-stranded nucleic acid molecules or fragments that can be subsequently removed from the GEM environment and amplified for library construction, including the addition of adaptor sequences for downstream sequencing. In one non-limiting example of the various embodiments herein, each such in-GEM 10× barcoded single-stranded nucleic acid molecule or fragment can include a unique molecular identifier (UMI), a unique 10× barcode, a Read 1 sequencing primer sequence, and a fragment or insert derived from an RNA fragment of the cell, e.g., cDNA from an mRNA via reverse transcription. Additional adaptor sequence may be subsequently added to the in-GEM barcoded nucleic acid molecules after the GEMs are broken.

In various embodiments, after the in-GEM barcoding process, the GEMs 150 are broken and pooled barcoded nucleic acid molecules or fragments are recovered. The 10× barcoded nucleic acid molecules or fragments are released from the droplets, i.e., the GEMs 150, and processed in bulk to complete library preparation for sequencing, as described in detail below. In various embodiments, following the amplification process, leftover biochemical reagents can be removed from the post-GEM reaction mixture. In one embodiment of the disclosure, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

Library Construction

The workflow 100 provided in FIG. 1A further includes a library construction step. In the library construction step of workflow 100, a library 170 containing a plurality of double-stranded DNA molecules or fragments are generated. These double-stranded DNA molecules or fragments can be utilized for completing the subsequent sequencing step. Detail related to the library construction, in accordance with various embodiments disclosed herein, is provided below.

In accordance with various embodiments disclosed herein, an Illumina® P7 sequence and P5 sequence (adapter sequences), a Read 2 (Read 2N) sequencing primer sequence, and a sample index (SI) sequence(s) (e.g., i7 and/or i5) can be added during the library construction step via PCR to generate the library 170, which contains a plurality of double stranded DNA fragments. In accordance with various embodiments herein, the sample index sequences can each comprise of one or more oligonucleotides. In one embodiment, the sample index sequences can each comprise of four to eight or more oligonucleotides. In various embodiments, when analyzing the single cell sequencing data for a given sample, the reads associated with all four of the oligonucleotides in the sample index can be combined for identification of a sample. Accordingly, in one non-limiting example, the final single cell gene expression analysis sequencing libraries contain sequencer compatible double-stranded DNA fragments containing the P5 and P7 sequences used in Illumina® bridge amplification, sample index (SI) sequence(s) (e.g., i7 and/or i5), a unique 10× barcode sequence, and Read 1 and Read 2 sequencing primer sequences.

Various embodiments of single cell sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

Targeted Gene Enrichment by Hybridization Capture

FIG. 1B depicts an example of a workflow for generating a targeted sequencing library using a hybridization capture approach. As shown, step 153 starts with obtaining a library of double stranded barcoded nucleic acid molecules from single cells (e.g., by partitioning single cells into droplets or wells with barcoding reagents including beads having nucleic acid barcode molecules) is denatured to provide single stranded molecules in step 154. To generate a targeted library using the single stranded molecules, a plurality of oligonucleotide probes designed to cover a panel of selected genes is provided. Each gene in the panel is represented by a plurality of labeled (e.g., biotinylated) oligonucleotide probes, which is allowed to hybridize to the single stranded molecules in step 155 to enrich for genes of interest (e.g. Target 1 and Target 2). To allow for capture, step 155 further includes the addition of supports (e.g., beads) that comprise a molecule having affinity for the labels on each labeled oligonucleotide probe. In one embodiment, the oligonucleotide label comprises biotin and the supports comprise streptavidin beads. Following hybridization capture, cleanup steps 156 and 157 are performed (e.g., one or more washing steps to remove unhybridized or off-target library fragments). Captured library fragments are then subjected to nucleic acid extension/amplification to generate a final targeted library for sequencing in step 158. This workflow allows the generation of targeted libraries from gene expression assays. In general, this workflow may be used to enrich any library of fragments having inserts or targets (light gray bar regions) that represent genes, e.g., cDNA transcribed from mRNA of single cells. It should be appreciated, however, that although the description above describes targeted gene enrichment through the use of hybridization capture probes, the methods disclosed herein can also work with other targeted gene enrichment techniques.

The workflow 100 provided in FIG. 1 further includes a sequencing step. In this step, the library 170 can be sequenced to generate a plurality of sequencing data 180. The fully constructed library 170 can be sequenced according to a suitable sequencing technology, such as a next-generation sequencing protocol, to generate the sequencing data 180. In various embodiments, the next-generation sequencing protocol utilizes the Illumina® sequencer for generating the sequencing data. It is understood that other next-generation sequencing protocols, platforms, and sequencers such as, e.g., MiSeq™, NextSeq™ 500/550 (High Output), HiSeq 2500™ (Rapid Run), HiSeg™ 3000/4000, and NovaSeg™, can be also used with various embodiments herein.

Sequencing Data Input and Data Analysis Workflow

The workflow 100 provided in FIG. 1 further includes a sequencing data analysis workflow 190. With the sequencing data 180 in hand, the data can then be output, as desired, and used as an input data 185 for the downstream sequencing data analysis workflow 190 for targeted gene expression analysis, in accordance with various embodiments herein. Sequencing the single cell libraries produces standard output sequences (also referred to as the “sequencing data”, “sequence data”, or the “sequence output data”) that can then be used as the input data 185, in accordance with various embodiments herein. The sequence data contains sequenced fragments (also interchangeably referred to as “fragment sequence reads”, “sequencing reads” or “reads”), which in various embodiments include RNA sequences of the targeted RNA fragments containing the associated 10× barcode sequences, adapter sequences, and primer oligo sequences.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. One example of a software tool that can process and input the sequencing data for downstream data analysis workflow is the cellranger-atac mkfastq tool within the Cell Ranger™ Targeted Gene Expression analysis pipeline (or the scRNA equivalent Cell Ranger™ analysis tool). It is understood that, various systems and methods with the embodiments herein are contemplated that can be employed to independently analyze the inputted single cell targeted gene expression analysis sequencing data for studying cellular gene expression, in accordance with various embodiments.

### Gene Expression Analysis Workflow

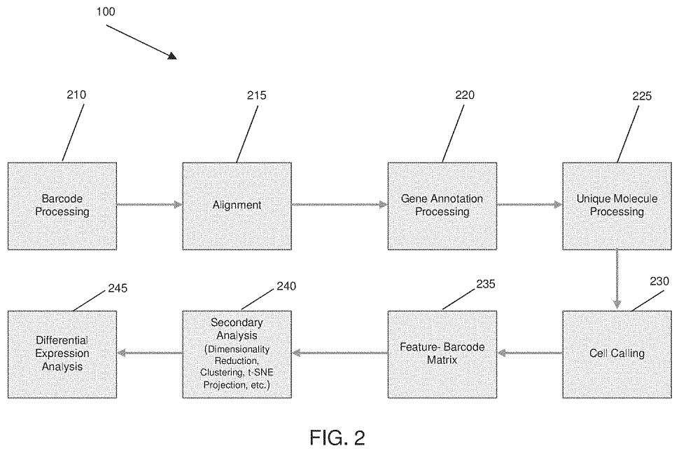

In accordance with various embodiments, a general schematic workflow is provided in FIG. 2 to illustrate a non-limiting example process of a sequencing data analysis workflow for gene expression analysis. The workflow can include various combinations of features, whether it be more or less features than that illustrated in FIG. 2. As such, FIG. 2 simply illustrates one example of a possible workflow for conducting gene expression analysis.

FIG. 2 provides a schematic workflow 200 for conducting gene expression analysis. It should be appreciated that the methodologies described in the workflow 200 of FIG. 2 and accompanying descriptions can be implemented independently of the methodologies for generating single cell sequencing data described in general. Therefore, FIG. 2 can be implemented independently of a sequencing data generating workflow as long as it is capable of sufficiently analyzing single cell sequencing data sets for gene expression analysis.

Moreover, the data analysis workflow can include one or more of the analysis steps illustrated in FIG. 2. Not all the steps within the disclosure of FIG. 2 need to be utilized as a group. Therefore, some of the steps within FIG. 2 are capable of independently performing the necessary data analysis as part of the various embodiments disclosed herein. Accordingly, it is understood that, certain steps within the disclosure can be used either independently or in combination with other steps within the disclosure, while certain other steps within the disclosure can only be used in combination with certain other steps within the disclosure. Further, one or more of the steps or filters described below, presumably defaulted to be utilized as part of the computational pipeline, can also not be utilized per user input. It is understood that the reverse is also contemplated. It is further understood that additional steps for analyzing the generated sequencing data are also contemplated as part of the computational pipeline within the disclosure.

Barcode Processing

The workflow 200 can comprise, at step 210, processing the barcodes in the single cell sequencing data set for fixing the occasional sequencing error in the barcodes so that the sequenced fragments can be associated with the original barcodes, thus improving the data quality. Detail related to the barcode processing and correction as part of the various embodiments disclosed herein is provided below.

In accordance with various embodiments, the barcode sequence can be between about 2 bp to about 25 bp. In accordance with various other embodiments, the barcode sequence can be between about 5 bp and 20 bp. In accordance with various preferred embodiments, the barcode sequence can be between about 10 bp and 16 bp. The length of the barcode sequence can affect the number of unique barcodes present in the sequencing library. Accordingly, it is understood that barcode sequences shorter than 10 bp can be selected in accordance with various embodiments herein, provided that the read sequence data from multiple cells are not associated with the same barcode because of severe lack of diversity caused by a shorter length of the barcode sequence. The barcode sequence can be obtained from the “12” index read and is read as part of the 12 reaction. Accordingly, it is understood that barcode sequences longer than 16 bp can be selected in accordance with various embodiments herein, provided that the barcode sequence length is within the limits of the 12 index read and reaction, and that it can be sequenced on a sequencer within the various embodiments herein. The barcode processing step can include checking each barcode sequence against a “whitelist” of correct barcode sequences. The barcode processing step can further include counting the frequency of each whitelist barcode. The barcode processing step can also include various barcode correction steps as part of the various embodiments disclosed herein. For example, one may attempt to correct the barcodes that are not included on the whitelist by finding all the whitelisted barcodes that are within 2 differences (Hamming distance <=2) of the observed sequence, and then scoring them based on the abundance of that barcode in the read data and quality value of the incorrect bases. As another example, an observed barcode that is not present in the whitelist can be corrected to a whitelist barcode if it has >90% probability of being the real barcode based.

Alignment

The workflow 200 can comprise, at step 215, aligning the read sequences (also referred to as the “reads”) to a reference sequence. In the alignment step of the various embodiments herein, a reference-based analysis is performed by aligning the read sequences (also referred to as the “reads”) to a reference sequence. The reference sequence for the various embodiments herein can include the reference genome sequence and its associated genome annotation, which includes gene and transcript coordinates. The genome sequences and annotations of various embodiments herein can be obtained from reputable, well-established consortia, including but not limited to NCBI, GENCODE, Ensembl, and ENCODE. In various embodiments, the reference sequence can include single species and/or multi-species reference sequences. In various embodiments, systems and methods within the disclosure can also provide pre-built single and multi-species reference sequences. In various embodiments, the pre-built reference sequences can include information and files related to regulatory regions including, but not limited to, annotation of promoter, enhancer, CTCF binding sites, and DNase hypersensitivity sites. In various embodiments, systems and methods within the disclosure can also provide building custom reference sequences that are not pre-built.

The alignment step may further include various sub-steps. For example, in various embodiments, the alignment step may include a sub-step that trims off the adapter, primer oligo sequence, or both in the read sequence before the read sequence is aligned to the reference genome. In various embodiments, the 3′ end of a read (i.e., the end of the read sequence) is presumed to contain the reverse complement of the primer sequence if the read sequence length is greater than the length of the genomic fragment. In such an event, the alignment step within various embodiments herein may include a sub-step that can identify the reverse complement of the primer sequence at the end of each read and trims off such sequence from the read sequence before the read sequence is aligned to the reference genome. In various embodiments, the cutadapt tool can be used to identify and trim off the reverse complement of the primer sequence from the read sequence prior to alignment.

The trimmed read-pairs described above can then be aligned to a specified genome reference using various methods in accordance with various embodiments herein. For example, in various embodiments, the Burrow-Wheeler Aligner (BWA-MEM) with default parameters can be used to align all the trimmed read-pairs to a specified reference. Examples of algorithm parameters and their corresponding default values are provided as follows: number of threads (default value of 1), minimum seed length (default value of 19), band width for banded alignment (default value of 100), off-diagonal X-drop-off (default value of 100), look for internal seeds inside a seed longer than {−k}*FLOAT (default value of 1.5, seed occurrence for the 3rd round seeding (default value of 20), skip seeds with more than INT occurrences (default value of 500), drop chains shorter than FLOAT fraction of the longest overlapping chain (default value of 0.5), discard a chain if seeded bases shorter than INT (default value of 0), perform at most INT rounds of mate rescues for each read (default value of 50), skip mate rescue, and skip pairing (mate rescue performed unless skip mate rescue parameter is also in use). Examples of scoring parameters/options and their corresponding default values are provided as follows: score for a sequence match (default value of 1), penalty for a mismatch (default value of 4), gap open penalties for deletions and insertions (default value of 6 and 6 respectively), gap extension penalty (default value of 1 and 1 respectively), penalty for 5′- and 3′-end clipping (default value of 5 and 5 respectively), penalty for an unpaired read pair (default value of 17), and read type. Examples of input/output options are as follows: smart pairing, read group header line (such as ‘@RG\tID:foo\tSM:bar’), insert STR to header if it starts with @ (or insert lines in FILE), sam file to output results to, treat ALT contigs as part of the primary assembly (i.e. ignore <idxbase>.alt file), for split alignment, take the alignment with the smallest coordinate as primary, do not modify mapQ of supplementary alignments, process INT input bases in each batch regardless of nThreads (for reproducibility), verbosity level (1=error, 2=warning, 3=message, 4+=debugging), minimum score to output (default value of 30), if there are <INT hits with score >80% of the max score, output all in XA, output all alignments for SE or unpaired PE, append FASTA/FASTQ comment to SAM output, output the reference FASTA header in the XR tag, use soft clipping for supplementary alignments, mark shorter split hits as secondary. Another example scoring option is to specify the mean, standard deviation (10% of the mean if absent), max (4 sigma from the mean if absent) and min of the insert size distribution.

In accordance with various embodiments, an aligner such as, for example, STAR (or other splice-aware aligners), to align of reads to the genome. Transcript annotation GTF (General Transcription Factors), for example, can be used to bucket the reads into exonic, intronic, and intergenic, and by whether the reads align to the genome. A read is exonic if at least 50% of it intersects an exon, intronic if it is non-exonic and intersects an intron, and intergenic otherwise. For reads that align to a single exonic locus but also align to one or more non-exonic loci, the exonic locus can be prioritized and the read can be considered to be confidently mapped to the exonic locus with mapping quality score (MAPQ) of 255.

For exonic reads, various embodiments herein can align the reads to annotated transcripts to look for compatibility. A read that is compatible with the exons of an annotated transcript, and aligned to the same strand, is considered mapped to the transcriptome. If the read is compatible with a single gene annotation, it is considered uniquely (confidently) mapped to the transcriptome. These confidently mapped reads are the only ones considered for UMI counting.

Various embodiments herein can be configured to correct for sequencing errors in the UMI sequences, before UMI counting. Reads that were confidently mapped to the transcriptome can be placed into groups that share the same barcode, UMI, and gene annotation. If two groups of reads have the same barcode and gene, but their UMIs differ by a single base (i.e., are Hamming distance 1 apart), then one of the UMIs was likely introduced by a substitution error in sequencing. In this case, the UMI of the less-supported read group is corrected to the UMI with higher support.

After grouping the reads by barcode, UMI (possibly corrected), and gene annotation, if two or more groups of reads have the same barcode and UMI, but different gene annotations, the gene annotation with the most supporting reads is kept for UMI counting, and the other read groups can be discarded. In case of a tie for maximal read support, all read groups can be discarded, as the gene cannot be confidently assigned.

After these two filtering steps, each observed barcode, UMI, gene combination is recorded as a UMI count in an unfiltered feature-barcode matrix, which contains every barcode from fixed list of known-good barcode sequences. This includes background and cell associated barcodes. The number of reads supporting each counted UMI is also recorded in the molecule info file.

Gene Annotation Processing

The workflow 200 can comprise, at step 220, annotating the individual RNA fragment reads as exonic, intronic, intergenic, and by whether they align to the reference genome with high confidence. In various embodiments, a fragment read is annotated as exonic if at least a portion of the fragment intersects an exon. In various embodiments, a fragment read is annotated as intronic if it is non-exonic and intersects an intron. The annotation process can be determined by the alignment method and its parameters/settings as performed, for example, using the STAR aligner.

Unique Molecule Processing

At step 225, in accordance with various embodiments, to better identify certain subpopulations such as for example, low RNA content cells, a unique molecule processing step can be performed prior to cell calling. For low RNA content cells, such a step is important, particularly when low RNA content cells are mixed into a population of high RNA content cells. The unique molecule processing can include a high content (e.g., RNA content) capture step and a low content capture step.

In the high content capture step, the processing can include using a cutoff based on total UMI counts of each barcode to identify cells. This step thus can identify the primary mode of high RNA content cells. This step can include, for example, receiving an expected number of recovered cells, N. With m representing the 99th percentile of the top N barcodes by total UMI counts, all barcodes whose total UMI counts exceed m/10 can be called as cells in the first pass.

In the low content capture step, the profile (e.g., RNA profile) of each remaining barcode is used to determine if it is an “empty” or a cell containing partition. This second step captures low RNA content cells whose total UMI counts may be similar to empty partitions. This step can include creating a model (or background model) of the profile (e.g., RNA profile) of selected barcodes, whereby the model can be a multinomial distribution over genes. The model can use a smoothing method (e.g., Good-Turing frequency estimation smoothing) to provide a non-zero model estimate for genes that were not observed in the representative empty GEM set. Finally, the RNA profile of each barcode not called as a cell in the first step is compared to the background model. Barcodes whose RNA profile strongly disagrees with the background model are added to the set of positive cell calls. This second step identifies cells that are clearly distinguishable from the profile of empty GEMs, even though they may have much lower RNA content than the largest cells in the experiment.

Cell Calling

The workflow 200 can comprise, at step 230, a cell calling analysis that includes associating a subset of barcodes observed in the library to the cells loaded from the sample. Identification of these cell barcodes can allow one to then analyze the variation in data at a single cell resolution. The process may further include correction of gel bead artifacts, such as gel bead multiples (where a cell shares more than one barcoded gel bead) and barcode multiplets (which occurs when a cell associated gel bead has more than one barcode). In some embodiments, the steps associated with cell calling and correction of gel bead artifacts are utilized together for performing the necessary analysis as part of the various embodiments herein.

In accordance with various embodiments, the record of mapped high-quality fragments or reads that passed all the filters of the various embodiments disclosed in the steps above and may be indicated as a fragment in the fragment file (e.g., a .csv file comprising the high-quality fragment information) or another designation based on the fragment source, may be recorded. With the genes, features or peaks determined in the calling step disclosed herein, the number of fragments that overlap any regions of interest (e.g. genes, features, or peaks), for each barcode, can be utilized to separate the signal from noise, i.e., to separate barcodes associated with cells from non-cell barcodes. It is to be understood that such method of separation of signal from noise works better in practice as compared to naively using the number of fragments per barcode.

Various methods, in accordance with various embodiments herein, can be employed to for cell calling. In various embodiments, the cell calling can be performed in two steps. In the first step of cell calling of the various embodiments herein, the barcodes that have a fraction of fragments overlapping called genes, features, or peaks lower than the fraction of region of interest in genes, features, or peaks are identified. When this first step is employed in the cell calling process of the various embodiments herein, the genes, features, or peaks may be padded by 2000 bp on both sides so as to account for the fragment length for this calculation.

Feature-Barcode Matrix

The workflow 200 can comprise, at step 235, generating a feature-barcode matrix that summarizes that gene expression counts per each cell. The feature-barcode matrix can include only detected cellular barcodes. The generation of the feature-barcode matrix can involve compiling the valid non-filtered UMI counts per gene (e.g., output from the ‘Unique Molecule Processing’ step discussed herein) from each cell-associated barcode (e.g., output from the ‘Cell Calling step discussed above) together into the final output count matrix, which can then be used for downstream analysis steps.

Secondary Analysis

The workflow 200 can comprise, at step 240, various dimensionality reduction, clustering and t-SNE projection tools. Dimensionality reduction tools of the various embodiments herein are utilized to reduce the number of random variables under consideration by obtaining a set of principal variables. In accordance with various embodiments herein, clustering tools can be utilized to assign objects of the various embodiments herein to homogeneous groups (called clusters) while ensuring that objects in different groups are not similar. T-SNE projection tools of the various embodiments herein can include an algorithm for visualization of the data of the various embodiments herein. In accordance with various embodiments, systems and methods within the disclosure can further include dimensionality reduction, clustering and t-SNE projection tools. In some embodiments, the analysis associated with dimensionality reduction, clustering, and t-SNE projection for visualization are utilized together for performing the necessary analysis as part of the various embodiments herein. Various analysis tools for dimensionality reduction such as Principal Component Analysis (PCA), Latent Semantic Analysis (LSA), and Probabilistic Latent Semantic Analysis (PLSA), clustering, and t-SNE projection for visualization that allow one to group and compare a population of cells with another, detail related to which are provided below.

Biological discovery is often aided by visualization tools that allow one to group and compare a population of cells with another. To enable such discovery, various visualization methods within the various embodiments herein, e.g., visualization methods within the Cell Ranger™ Targeted Gene Expression analysis pipeline, can be employed. In various embodiments, such visualization methods can include clustering and T-distributed Stochastic Neighbor Embedding (t-SNE) projection tools.

The systems and methods within the disclosure are directed to identifying differential gene expression. As the data is sparse at single cell resolution, dimensionality reduction in accordance with various embodiments herein can be performed to cast the data into a lower dimensional space.

Various systems and methods of various embodiments herein, e.g., systems and methods of the Cell Ranger™ Targeted Gene Expression analysis pipeline, can be utilized to support dimensionality reduction. Various methods, such as Principal Component Analysis (PCA), Latent Semantic Analysis (LSA), and Probabilistic Latent Semantic Analysis (PLSA), can support dimensionality reduction in accordance with various embodiments herein. In various embodiments, before clustering the cells, LSA is run on the normalized filtered barcode matrix to reduce the number of feature or gene dimensions. This produces a projection of each cell onto the first N components (default N=15). Thus, various embodiments, the adopted default method of dimensionality reduction is LSA. In other embodiments within the disclosure, users may alternatively choose PCA or PLSA to perform dimensionality reduction in the pipeline. Accordingly, users can specify which dimensionality reduction method to use by providing a dimensionality reduction parameter. In various embodiments, the dimensionality reduction parameter can be “--dim-reduce=<Method>”, which can be specified to the various embodiments herein, e.g., the Cell Ranger™ Targeted Gene Expression pipeline. In accordance with various embodiments herein, each dimensionality reduction method within the disclosure can have an associated data normalization technique that is used prior to the dimensionality reduction step.

In accordance with various embodiments herein, a collection of clustering methods within the disclosure can be employed to accept the dimensionality reduced data. In various embodiments, an optimized implementation of the Barnes Hut TSNE algorithm can be employed to project the dimensionality reduced data into 2-D t-SNE space. In accordance with various embodiments herein, the number of dimensions can be fixed to 15. In various embodiments within the disclosure, it has been observed that fixing the number of dimensions 15 can sufficiently separate clusters visually and in a biologically meaningful way when tested on peripheral blood mononuclear cells (PBMCs). It is understood that other number of dimensions can be fixed in accordance with various embodiments herein. In various embodiments within the disclosure, the number of dimensions (i.e., dimensions of the matrix) can be fixed at a number less than the number of cell-barcodes and the number of genes, features, or peaks. In various embodiments, the number of dimensions can be at least 15. In various embodiments, the number of dimensions can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100. It can be understood that higher numbers of dimensions can be computationally costly. Accordingly, computational costs can be determinative of the number of dimensions used. More detail regarding the various methods dimensionality reduction methods described above is provided below.

PCA

When PCA is the dimensionality reduction method of the various embodiments herein, the data is normalized to median cut site counts per barcode and log-transformed. In various embodiments, a fast, scalable and memory efficient implementation of IRLBA (Augmented, Implicitly Restarted Lanczos Bidiagonalization Algorithm) can be used that allows in-place centering and feature scaling and produces the transformed matrix along with the principal components (PC) and singular values encoding the variance explained by each PC. When PCA is the default dimensionality reduction method of the various embodiments herein, k-means clustering can be used. In various embodiments, k-means clustering produces 2 to 10 clusters for visualization and analysis. In another embodiment within the disclosure, a k-nearest neighbors graph-based clustering method is also provided via community detection using a modularity optimization algorithm. In various embodiments, the modularity optimization algorithm is louvain modularity optimization algorithm. The transformed matrix of the various embodiments herein, can be operated on by the t-SNE algorithm with default parameters (e.g., tsne_input_pcs, tsne_perplexity, tsne_theta, tsne_max_dims, tsne_max_iter, tsne_stop_lying_iter, and tsne_mom_switch_iter) and can provide 2-D coordinates for each barcode for visualization with various embodiments herein.

Below is a summary of default parameters of t-SNE algorithm in accordance with various embodiments.

| | | Default | | |
|---|---|---|---|---|
| Parameter | Type | Value | Recommended Range | Description |
| tsne_input_pcs | int | null | e be set higher than the | Subset to top N |
| | | | num_comps parameter, which | principal components |
| | | | is N principal components for | for TSNE. |
| | | | LSA/PCA/PLSA. | |
| tsne_perplexity | int | 30 | 30-50 | TSNE perplexity |
| | | | | parameter. |
| tsne_theta | float | 0.5 | Between 0 and 1. | TSNE theta parameter. |
| tsne_max_dims | int | 2 | 2 or 3. | Maximum number of |
| | | | | TSNE output |
| | | | | dimensions. |
| tsne_max_iter | int | 1000 | 1000-10000 | Number of total TSNE |
| | | | | iterations. |
| tsne_stop_lying_iter | int | 250 | Cannot be set higher than | Iteration at which |
| | | | tsne_max_iter. | TSNE learning rate is |
| | | | | reduced. |
| tsne_mom_switch_iter | int | 250 | Cannot be set higher than | Iteration at which |
| | | | tsne_max_iter. | TSNE momentum is |
| | | | | reduced. |

LSA

In accordance with various embodiments herein, the data can be normalized via the inverse-document frequency (idf) transformer, where each gene, feature, or peak count can be scaled by the log of the ratio of the number of barcodes in the matrix and the number of barcodes where the gene, feature, or peak has a non-zero count. This normalization can provide greater weight to counts in genes, features, or peaks that occur in fewer barcodes. In some embodiments within the disclosure, singular value decomposition (SVD) can be performed on this normalized matrix using IRLBA without scaling or centering, to produce the transformed matrix in lower dimensional space, as well as the components and the singular values signifying the importance of each component. In some embodiments within the disclosure, prior to clustering, normalization to depth can be performed by scaling each barcode data point to unit L2-norm in the lower dimensional space. It has been observed that the combination of these normalization techniques obviates the need to remove the first component in accordance with various embodiments herein. When LSA is the dimensionality reduction method of the various embodiments herein, a spherical k-means clustering can be provided that produces 2 to 10 clusters for downstream analysis. It has been observed that spherical k-means can perform better than plain k-means, by identifying clusters via k-means on L2-normalized data that can live on the spherical manifold. Accordingly, spherical k-means can be suitable to cluster large-scale datasets from aggregation runs, as described in detail below. In another embodiment within the disclosure, and similar to PCA, a graph-based clustering can be provided and visualized via t-SNE. However, similar to spherical k-means clustering, the data can be normalized to unit norm before performing graph-based clustering and t-SNE projection.

PLSA

PLSA is a special type of non-negative matrix factorization, with roots in Natural Language Processing. When PLSA is the dimensionality reduction method of the various embodiments herein, the KL-divergence between the empirically determined probability of observing a gene, feature, or peak in a barcode and the lower rank approximation to it is minimized, via an Expectation-Maximization algorithm. In various embodiments, the data is not normalized prior to dimensionality reduction via PLSA. Similar to LSA and PCA, a transformed matrix, component vectors, and a set of values explaining the importance of each component can be produced in accordance with various embodiments herein. PLSA can offer natural interpretation of the components and the transformed matrix of the various embodiments herein. In accordance with various embodiments herein, each component can be interpreted as a hidden topic and the transformed matrix can simply be the probability of observing a barcode from a given topic (i.e., Prob(barcode|topic)). In accordance with various embodiments herein, the component vectors can be the probability of observing a gene, feature, or peak from a given topic (i.e., (Prob(peak|topic) or (Prob(gene|topic) or (Prob(feature|topic)) and the counterpart to singular values of LSA/PCA can simply be the probability of each topic (i.e., (Prob(topic)) observed in the data of various embodiments herein. Similar to LSA, the transformed matrix for PLSA can be normalized to unit L2-norm. In various embodiments, spherical k-means clustering can be then be performed to produce 2 to 10 clusters. In another embodiment within the disclosure, graph-based clustering can also be performed. The transformed matrix of the various embodiments herein, can then be visualized via t-SNE. It is understood that while PLSA offers great advantages in interpretability of the lower dimensional space, it is appreciably slower than both PCA and LSA and does not scale well beyond 20 components on large datasets. To ameliorate this to some extent, in various embodiments, the in-house implementation of PLSA can be multithreaded (4 threads on compute cluster) and written and compiled in C++. To ensure a reasonable run time, in various embodiments, the algorithm can be capped at 3000 iterations in the event it does not converge first. It is understood that other number of dimensions can be fixed in accordance with various embodiments herein.

It is understood that various clustering methods including, but not limited to, K-Means clustering, affinity propagation, mean-shift, spectral clustering, Ward hierarchical clustering, agglomerative clustering, DBSCAN, OPTICS, Gaussian mixture models, Birch clustering, and k-medoids clustering, and visualization approaches can be utilized in accordance with various embodiments herein. It is understood that each clustering method may have various tradeoffs. Accordingly, in various embodiments, selection of a clustering method can be made based on whether the clusters make biological sense with known, well-studied sample types (e.g., PBMCs), i.e., whether the clusters generated using a particular clustering method make sense with validation on known biology. Below is a non-limiting summary of dimensionality reduction techniques and associated clustering and visualization approaches in accordance with various embodiments herein.

| Dimensionality Reduction | Clustering | Visualization |
|---|---|---|
| PCA | K-means, graph-clustering | TSNE |
| LSA | Spherical k-means, | TSNE |
| | graph-clustering | |
| PLSA | Spherical k-means, | TSNE |
| | graph-clustering | |

Differential Expression Analysis

In accordance with various embodiments, the workflow 200 can comprise, at step 245, a differential expression analysis that performs differential analysis to identify genes whose expression is specific to each cluster, Cell Ranger tests, for each gene and each cluster, whether the in-cluster mean differs from the out-of-cluster mean.

To find differentially expressed genes between groups of cells, systems and methods disclosed herein can use the quick and simple method sSeq (Yu, D., Huber, W. & Vitek, O. “Shrinkage estimation of dispersion in Negative Binomial models for RNA-seq experiments with small sample size” Bioinformatics 29, 1275-1282 (2013)), which employs a negative binomial exact test. When the counts become large, systems and methods disclosed herein switches to the fast asymptotic beta test used in edgeR (Robinson, M. D. & Smyth, G. K. “Small-sample estimation of negative binomial dispersion, with applications to SAGE data” Biostatistics 9, 321-332 (2007)). For each cluster, the algorithm is run on that cluster versus all other cells, yielding a list of genes that are differentially expressed in that cluster relative to the rest of the sample.

The implementation of the systems and methods disclosed herein may incorporate, or even differs from that in the sSeq paper where the authors recommend using DESeq's geometric mean-based definition of library size. The systems and methods disclosed herein can, instead, compute relative library size as the total UMI counts for each cell divided by the median UMI counts per cell. As with sSeq, normalization is implicit in that the per-cell library-size parameter is incorporated as a factor in the exact-test probability calculations.

Targeted Unique Molecule Filtering

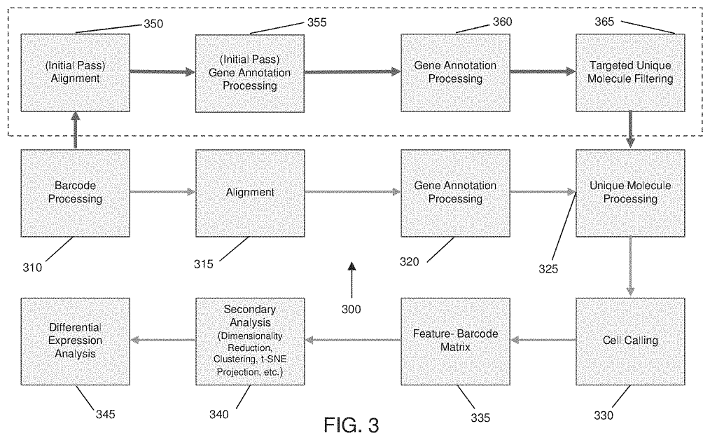

Referring now to FIG. 3, an exemplary flowchart is provided showing a workflow 300 for conducting for targeted gene expression analysis, in accordance with various embodiments. Steps 310 to 345 are substantially similar to steps 210 to 245 of workflow 200, illustrated in FIG. 2. The foregoing will discuss primarily steps 350 to 365, which assist in enabling for workflow 300 to execute a targeted gene expressions analysis.

In step 350, subsequent to barcode processing step 310, and concurrent with alignment step 315, initial pass alignment step 350 can be conducted. The alignment conducted in step 350 can be performed using many of the methods and algorithms discussed above in regard to alignment step 215/315. As compared to alignment step 215/315, which covers the entire data set, alignment step 350 can apply specifically to a subset of the full dataset (i.e., a randomly selected set of barcodes or a subset of barcodes specific to targeted nature of the gene expression analysis of FIG. 3). For example, for data that is subset to a random selection of 10% of the barcodes, that percentage can be chosen to ensure that at least 10 million reads are included in the subset (otherwise the 10% fraction can be increased to meet this limit). Note that while alignment step 350 is shown to be concurrent with alignment step 315, these steps can be considered independent. As such, initial pass alignment 350 (as well as the remaining steps 355-365) can be completed before the overall analysis steps 315 and 320 are performed.

In step 355, an initial pass gene annotation processing step occurs, concurrently with gene annotation step 320. The processing conducted in step 355 can be performed using many of the methods and algorithms discussed above regarding processing step 220/320. As compared to processing step 220/320, which covers the entire data set, processing step 355 can apply specifically to a subset of the full dataset (i.e., a randomly selected set of barcodes or a subset of barcodes specific to targeted nature of the gene expression analysis of FIG. 3). Note that while processing step 355 is shown to be concurrent with processing step 320, these steps can be considered independent. As such, initial pass processing 355 can be completed before the overall analysis steps 315 and 320 are performed.

In step 360, a gene annotation process is conducted. This process is substantially similar to gene annotation step 320. The processing conducted in step 360 can be performed using many of the methods and algorithms discussed above regarding processing step 220/320. As stated above, while gene annotation processing step 360 is shown to be concurrent with processing step 320, these steps can be considered independent. As such, gene annotation processing step 360 can be completed before the overall analysis steps 315 and 320 are performed.

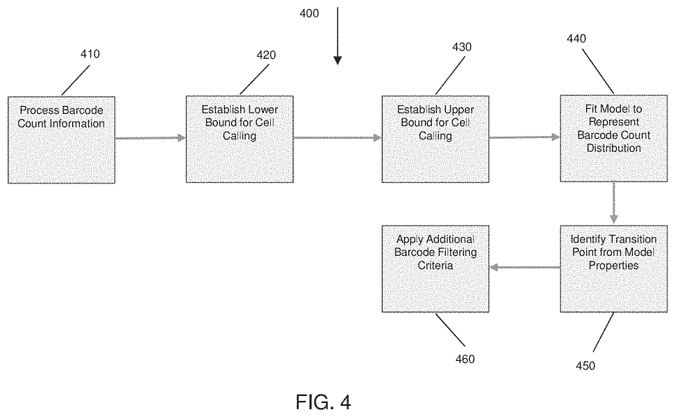

It should be noted that while flow 300 depicts the current architecture and implementation of the pipeline as having a split workflow, a split workflow is not required. In accordance with various embodiments, all the analysis steps and computations could be achieved in a single-path workflow. Further, in accordance with various embodiments, some of these steps can be removed or consolidated as needed, particularly given the multiple alignment, gene annotation, and unique molecule filtering steps that occur on workflow 300. For example, a single flow diagram could be similar to FIG. 2 with an example modification of adding a targeted unique molecule filtering step (see step 365 discussed below) after the unique molecule processing step 225/325 and before the cell calling step 230/330. This flow could use the total output from the original unique molecule processing step 225/325 step, then could calculate the parameters for targeted UMI filtering (discussed below with example FIG. 4), and could then revise the updated results after the filtering was performed before proceeding to the cell calling step.

In step 365, a targeted unique molecule filtering step is performed. As compared to predecessor alignment and gene annotation process steps 350 to 360, filtering step 365 employs some aspects of unique molecule processing step 225/325, but does not mirror step 225/325. For example, this step can first compute and tabulate the results in the way those steps are performed in step 225/325, but then do additional calculations and report back the filtering parameter threshold value which is used to filter out low-confidence molecules that would have otherwise been reported in the subsequent step 225/325.

At step 365, in accordance with various embodiments, to better identify certain subpopulations of cells such as for example, low RNA content cells, a targeted unique molecule processing step can be performed prior to cell calling. For low RNA content cells, such a step is important, particularly when low RNA content cells are mixed into a population of high RNA content cells. To do this, two analyses are conducted to identify two different cutoff points, a high (high count) and low (low count) cutoff. FIG. 4 provides an example workflow for this analysis. Examples of cells/samples can include, for example, PBMCs (Peripheral Blood Mononuclear Cells), cell lines, primary cell samples, dissociated tumor cells, primary neurons, fibroblasts, cardiomyocytes, cultured neurons, embryonic cell samples, samples comprising cells from multiple species, immune cells Isolated from tumor cells or patient samples, FACS-sorted cells, and isolated nuclei samples (which are not technically complete cells).

FIG. 4 provides a workflow 400 for targeted unique molecule filtering. In step 410, received barcode count information is processed. The results that are output by steps 225/325 can be are input into cell calling step 230/330, which corresponds to the estimated number of unique detected counts per gene (or molecule) for each barcode.

In step 420, a high count cut off value (or lower bound for cell calling as will be discussed later) is determined. To determine the high cutoff, the processing can include using a cutoff associated with total molecule counts/UMI counts of each cell/barcode to identify cells. This step can include, for example, receiving an expected number of recovered cells, N. With m representing count corresponding to the top 1^{st} percentile of the top N sorted cells/barcodes by total molecule counts/UMI counts, the count value of m/10 determines the high cutoff (or lower bound as will be discussed later) for subsequent cell calling. The percentile is a parameter that can be varied. For example, the percentile can range from about 0.5% to about 20%, from about 1% to about 10%, any iterative ranges in between, about 1%, and so on. Because of the possible interrelated nature of percentile and fold change parameter (e.g., the m/10 value for the denominator—by default equal to 10), an adjustment to the accompanying fold-change parameter can take place. For example, for an N value of 3000, the top 1^{st} percentile would be the 30^{th} ranked barcode by count. If that count were 500, the m/10 high cutoff would be a count of 50, with any barcodes exceeding that value being considered cells in this first analysis. The denominator is a parameter that can be varied. For example, the denominator can range from about 2 to about 50, about 3 to about 20, any iterative ranges in between, about 10, and so on.

In step 430, a low count cut off value (or upper bound for cell calling as will be discussed later) is determined. To determine the low cutoff, for all cells, additional barcodes are added to the analysis that did not qualify under the high cut off (lower bound) analysis discussed above. The number of additional barcodes can be varied. For example, up to 2,000 more barcodes can be added. The number of additional barcodes can range from about 5,000 to about 50,000 and any iterative ranges in between. Partly the choice of reasonable values can be affected by the number of recovered partitions normally recovered from each GEM-well lane per sample (e.g., approximately 90,000). The parameter may also vary depending on the number of input cells, which can be any number up to about 10,000, any number up to about 35,000, and so on. Note however that the cell number should not be limited to these values and should support increased numbers as needed based on the platform's capabilities.

To establish the upper bound, a minimum count value is provided or determined, and all additional barcodes are considered against that minimum count. If the additional barcodes have counts greater than or equal to the defined acceptable minimum count value, then the additional barcodes are considered in subsequent analysis. The minimum count value is variable. For example, the minimum count value can be 10 counts, which becomes the upper bound for (low count cut off value) targeted gene expression analysis. The minimum count can be in the range of about five to about 100, and any iterative range in between.

It should be noted that this method for determining upper and lower bounds is usable in different constructs. For example, it can be used for cell-calling involving non-targeted data. In that type of construct, the minimum count value for considering additional barcodes may be set to a different value higher than 10 counts, since non-targeted data includes counts from all genes, and therefore can retain a larger number of counts per cell. For example, the value could be about 500, could range from about 100 to about 500, or about 100 to 1000, or any iterative ranges in between.

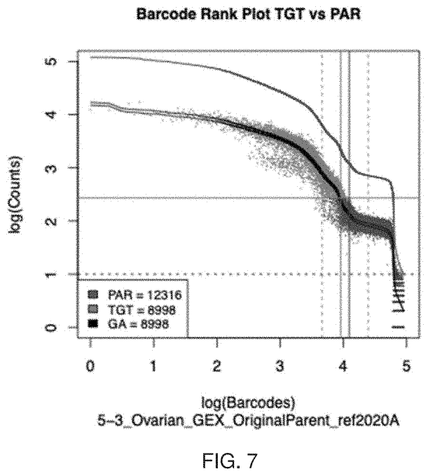

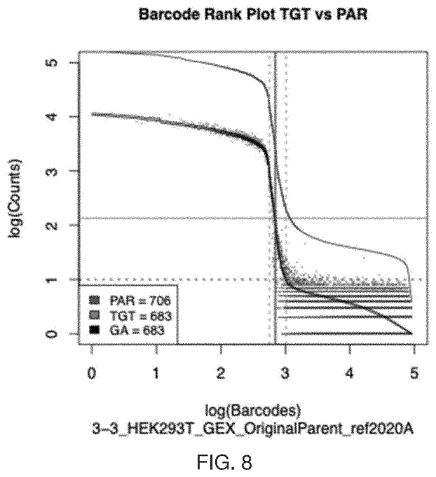

In step 440, a model is fit to the barcodes within the lower and upper bounds to represent barcode distribution. Examples of this model are provided in FIGS. 7 and 8, which illustrate barcode rank plots with the log of count values (e.g., UMI/cell counts) as a function of the log of the ranked barcodes. Referring to FIGS. 7 and 8, the left most dotted vertical line represents the lower bound and the right most dotted vertical line represents the upper bound, both discussed above.

In step 450, a transition point is identified from the model. This transition point can be determined, for example, using slope analysis on the log-transformed barcode-rank plot curve illustrated in FIGS. 7 and 8. Slope analysis can include, for example, mathematically determine the steepest slope on the log/log curve, which would represent the point where first derivative reaches global minimum value. This analysis can be done using a variety of methods. In FIGS. 7 and 8, for example, a cubic spline curve is fitted to the log-transformed barcode-rank plot, and the steepest slope is determined. On FIGS. 7 and 8, this point is visually identified by the intersection of the curve and the left vertical line located between the lower and upper bounds (the right vertical line is a reference line, independent of the analysis described herein). Other mathematical tools that can also be used include, for example, regression analysis (linear or non-linear), probability distribution fitting, smoothing, interpolation methods, trend estimation, least-squares estimation, function approximation, Loess fitting, etc.

At step 460, additional barcode filtering criteria can be applied. For targeted gene expression analysis, this can include applying a targeted filter threshold (i.e., UMI >0) to the targeted cell subset. As a result, this filter allows for the capturing of all targeted cells from to total cells that were gathered via the preceding steps of FIG. 4. This step is essentially where the filtering for targeted cell-calling can be applied. For non-targeted cell-calling, for example, such an exact step may not be needed. However, such a step 460 could be modified or expanded to potentially incorporate other criteria, such as, for example, whether or not the UMI counts were derived from a sufficient number of different genes, or to impose other criteria. For targeted data, the setting of UMI counts >0 is the absolute minimum, and could be increased to about 10 or about 20 minimum targeted counts (varies depending on, e.g., the target panel composition and sample type). Moreover, the criteria could be extended to not just include the total number of targeted counts to be above zero, but also to require, for example, that at least some number of targeted genes all have counts above zero or some other minimum value, which furthermore could vary per gene.

In various embodiments, methods are provided for identifying a subset of cells from a dataset for targeted gene expression analysis. The methods can be implemented via computer software or hardware. The methods can also be implemented on a computing device/system that can include a combination of engines for identifying a subset of cells from a dataset for targeted gene expression analysis. In various embodiments, the computing device/system can be communicatively connected to one or more of a data source, sample analyzer (e.g., a genomic sequence analyzer), and display device via a direct connection or through an internet connection.

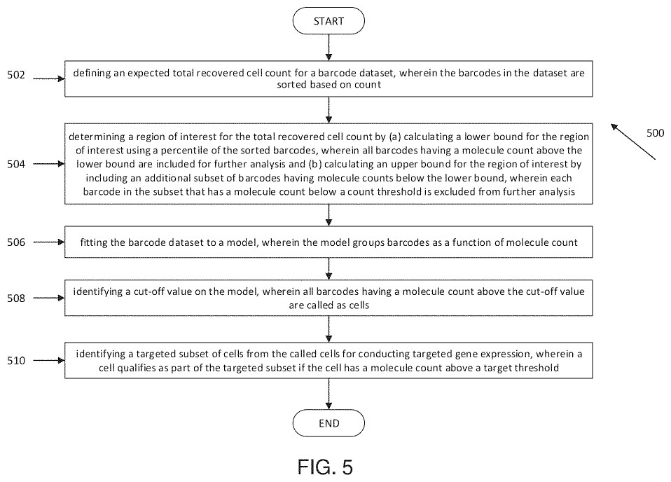

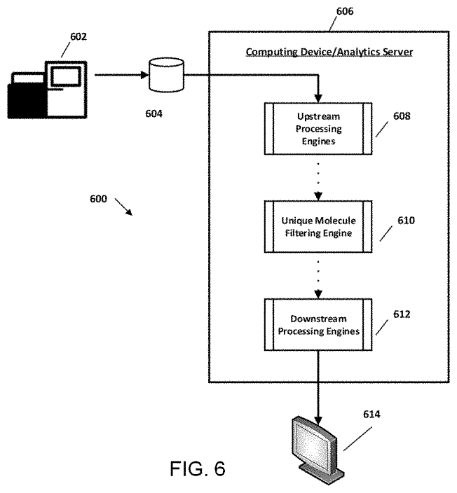

Referring now to FIG. 5 a flowchart illustrating a non-limiting example method 500 for identifying a subset of cells from a dataset for targeted gene expression analysis is disclosed, in accordance with various embodiments. The method can comprise, at step 502, defining an expected total recovered cell count for a barcode dataset, wherein the barcodes in the dataset are sorted based on count.

The method can comprise, at step 504, determining a region of interest (ROI) for the total recovered cell count. The ROI can be determined by calculating a lower bound for the region of interest using a percentile of the sorted barcodes, wherein all barcodes having a molecule count above the lower bound are included for further analysis. The ROI can further be determined by calculating an upper bound for the region of interest by including an additional subset of barcodes having molecule counts below the lower bound, wherein each barcode in the subset that has a molecule count below a count threshold is excluded from further analysis. In accordance with various embodiments, the percentile can be the 1^{st} percentile barcode of an expected number of cell-associated barcodes. The lower bound can be 10 percent of the count of the 1^{st} percentile barcode. The additional subset of barcodes can comprise about 2,000 barcodes. The count threshold can be about 10 counts.

The method can comprise, at step 506, fitting the barcode dataset to a model, wherein the model groups barcodes as a function of molecule count. In accordance with various embodiments, the model can be a plot of count as a function of ranked barcodes. The plot can be a log-transformed barcode-rank plot.

The method can comprise, at step 508, identifying a cut-off value on the model, wherein all barcodes having a molecule count above the cut-off value are called as cells. In accordance with various embodiments, the cut-off value on the model can be determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof. The cut-off value can be the point on the curve where the first derivative reaches a global minimum value

The method can comprise, at step 510, identifying a targeted subset of cells from the called cells for conducting targeted gene expression, wherein a cell qualifies as part of the targeted subset if the cell has a molecule count above a target threshold. In accordance with various embodiments, the target threshold is a count greater than zero.

In accordance with various embodiments, FIG. 6 illustrates a non-limiting example system for conducting targeted gene expression analysis, in accordance with various embodiments. The system 600 includes a genomic sequence analyzer 602, a data storage unit 604, a computing device/analytics server 606, and a display 614.

The genomic sequence analyzer 602 can be communicatively connected to the data storage unit 604 by way of a serial bus (if both form an integrated instrument platform) or by way of a network connection (if both are distributed/separate devices). The genomic sequence analyzer 602 can be configured to process, analyze and generate one or more genomic sequence datasets from a sample, such as the targeted gene expression fragment libraries of the various embodiments herein. Each fragment in the library includes an associated barcode and unique identifier sequence (i.e., UMI). In various embodiments, the genomic sequence analyzer 602 can be a next-generation sequencing platform or sequencer such as the Illumina® sequencer, MiSeq™, NextSeq™ 500/550 (High Output), HiSeq 2500™ (Rapid Run), HiSeg™ 3000/4000, and NovaSeq.

In various embodiments, the generated genomic sequence datasets can then be stored in the data storage unit 604 for subsequent processing. In various embodiments, one or more raw genomic sequence datasets can also be stored in the data storage unit 604 prior to processing and analyzing. Accordingly, in various embodiments, the data storage unit 604 can be configured to store one or more genomic sequence datasets, e.g., the genomic sequence datasets of the various embodiments herein that includes a plurality of fragment sequence reads with their associated barcodes and unique identifier sequences. In various embodiments, the processed and analyzed genomic sequence datasets can be fed to the computing device/analytics server 606 in real-time for further downstream analysis.

In various embodiments, the data storage unit 604 is communicatively connected to the computing device/analytics server 606. In various embodiments, the data storage unit 604 and the computing device/analytics server 606 can be part of an integrated apparatus. In various embodiments, the data storage unit 604 can be hosted by a different device than the computing device/analytics server 606. In various embodiments, the data storage unit 604 and the computing device/analytics server 606 can be part of a distributed network system. In various embodiments, the computing device/analytics server 606 can be communicatively connected to the data storage unit 604 via a network connection that can be either a “hardwired” physical network connection (e.g., Internet, LAN, WAN, VPN, etc.) or a wireless network connection (e.g., Wi-Fi, WLAN, etc.). In various embodiments, the computing device/analytics server 606 can be a workstation, mainframe computer, distributed computing node (part of a “cloud computing” or distributed networking system), personal computer, mobile device, etc.

In various embodiments, the computing device/analytics sever 606 is configured to host one or more upstream data processing engines 608, a Unique Molecule Filtering Engine 610, and one or more downstream data processing engines 612.

Examples of upstream data processing engines 608 can include, but are not limited to: alignment engine, cell barcode processing engine (for correcting sequencing barcode sequencing errors), alignment engine (for aligning the fragment sequence reads to a reference genome), annotation engine (for annotating each of the aligned fragment sequence reds with relevant information), etc.

The Unique Molecule Filtering Engine 610 can be configured to receive one or more genomic sequence datasets that are stored in the data storage unit 604. The genomic sequence datasets are comprised for a plurality of fragment sequence reads (generated from the sequencing of a fragment library, for example, a targeted gene expression fragment library), each with an associated barcode sequence and a unique identifier sequence (i.e., UMI). In various embodiments, the Unique Molecule Filtering Engine 610 can be configured to receive processed and analyzed genomic sequence datasets from the genomic sequence analyzer 602 in real-time.

In various embodiments, the Unique Molecule Filtering Engine 610 can be configured to identify a subset of cells from a dataset for targeted gene expression analysis.

In various embodiments, the engine 610 can be configured to define an expected total recovered cell count for a barcode dataset, wherein the barcodes in the dataset are sorted based on count.

In various embodiments, the engine 610 can be configured to determine a region of interest (ROI) for the total recovered cell count. The ROI can be determined by calculating a lower bound for the region of interest using a percentile of the sorted barcodes, wherein all barcodes having a molecule count above the lower bound are included for further analysis. The ROI can further be determined by calculating an upper bound for the region of interest by including an additional subset of barcodes having molecule counts below the lower bound, wherein each barcode in the subset that has a molecule count below a count threshold is excluded from further analysis. In accordance with various embodiments, the percentile can be the 1st percentile barcode of ranked barcodes. The lower bound can be 10 percent of the count of the 1st percentile barcode. The additional subset of barcodes can comprise about 2,000 barcodes. The count threshold can be about 10 counts.

In various embodiments, the engine 610 can be configured to fit the barcode dataset to a model, wherein the model groups barcodes as a function of molecule count. In accordance with various embodiments, the model can be a plot of count as a function of ranked barcodes. The plot can be a log-transformed barcode-rank plot.

In various embodiments, the engine 610 can be configured to identify a cut-off value on the model, wherein all barcodes having a molecule count above the cut-off value are called as cells. In accordance with various embodiments, the cut-off value on the model can be determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof. The cut-off value can be the point on the curve where the first derivative reaches a global minimum value

In various embodiments, the engine 610 can be configured to identify a targeted subset of cells from the called cells for conducting targeted gene expression, wherein a cell qualifies as part of the targeted subset if the cell has a molecule count above a target threshold. In accordance with various embodiments, the target threshold is a count greater than zero.

The identified subset of cell then be further processed by one or more downstream data processing engines 612 for the purposes of conducing targeted gene expression. Examples of downstream data processing engines 612 can include, but are not limited to: cell calling engine (for grouping fragment sequence reads as being from a unique cell), feature barcode matrix engine (for creating a feature barcode matrix), differential analysis engine (for identifying genes whose expression is specific to each cell cluster), etc.

After the downstream processing has been performed and an output of the results can be displayed as a result or summary on a display or client terminal 614 that is communicatively connected to the computing device/analytics server 606. In various embodiments, the display or client terminal 614 can be a thin client computing device. In various embodiments, the display or client terminal 614 can be a personal computing device having a web browser (e.g., INTERNET EXPLORER™, FIREFOX™, SAFARI™, etc.) that can be used to control the operation of the genomic sequence analyzer 602, data store 604, upstream data processing engines 608, Unique Molecule Filtering Engine 610, and the downstream data processing engines 612.

It should be appreciated that the various engines can be combined or collapsed into a single engine, component or module, depending on the requirements of the particular application or system architecture. In various embodiments engines 608/610/612 can comprise additional engines or components as needed by the particular application or system architecture.

### Examples

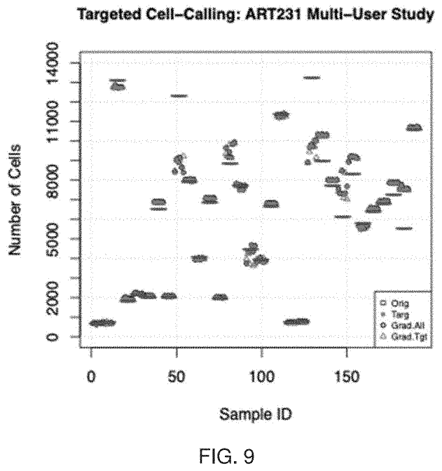

FIG. 9 is a chart depicting the number of identified cell-associated barcodes from a particular single-cell sample (plotted on the y-axis) versus an associated sample identifier index (plotted on the x-axis), in accordance with various embodiments. The Sample ID values on the x-axis are ordered such that each group of 6 consecutive values (numbered #1-6, #7-12, and so on) corresponds to 6 different targeted gene expression samples which were each derived from the same unique single-cell WTA input sample. The points plotted as hollow squares (“Orig”) (in blue) show the number of barcodes classified as cells for the original WTA input sample, analyzed using the standard cell-calling method from Cell Ranger 3.1, and are invariant across each set of 6 associated targeted samples per group. The points plotted as solid circles (“Targ”) (in red) show the number of barcodes classified as cells for each targeted gene expression sample, analyzed using targeted cell-calling method introduced for Cell Ranger 4.0. The points plotted as open circles (“Grad.All”) (in black) show the number of barcodes classified as cells for each targeted gene expression sample, analyzed using the new gradient cell-calling method, considering counts from all genes. The points plotted as open triangles (“Grad.Tgt”) (in red) show the number of barcodes classified as cells for each targeted gene expression sample, analyzed using the new gradient cell-calling method considering counts from only targeted genes.

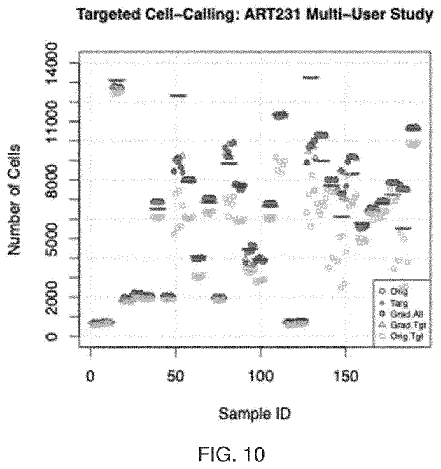

FIG. 10 is a chart depicting the number of identified cell-associated barcodes from a particular single-cell sample (plotted on the y-axis) versus an associated sample identifier index (plotted on the x-axis), plotted as described for FIG. 9, in accordance with various embodiments. Additional points plotted as open circles (“Orig.Tgt”) (in green) show the number of barcodes classified as cells for each targeted gene expression sample, analyzed using the standard cell-calling method (originally intended for non-targeted datasets) from Cell Ranger 3.1.

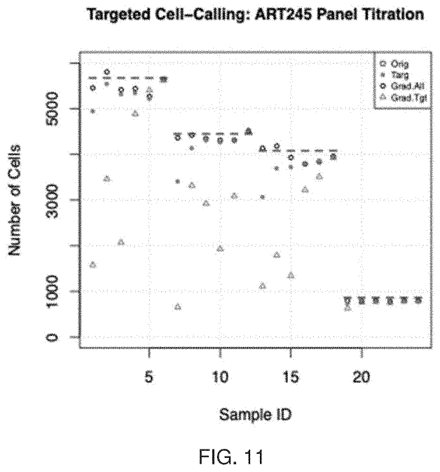

FIG. 11 is a chart depicting the number of identified cell-associated barcodes from a particular single-cell sample (plotted on the y-axis) versus an associated sample identifier index (plotted on the x-axis), plotted as described for FIG. 9, in accordance with various embodiments. Moreover, the targeted samples within each group of 6 reflect progressively increasing numbers of targeted genes that were included in the target gene panel for each sample.

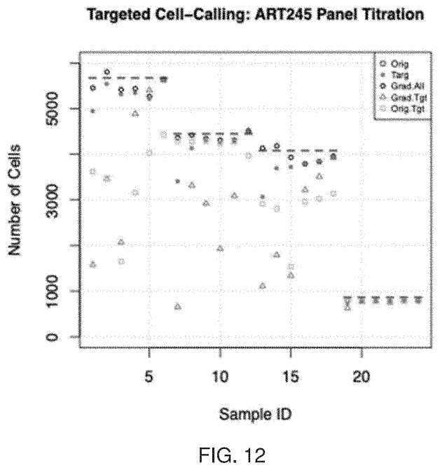

FIG. 12 is a chart depicting the number of identified cell-associated barcodes from a particular single-cell sample (plotted on the y-axis) versus an associated sample identifier index (plotted on the x-axis), plotted as described for FIG. 9, in accordance with various embodiments. Additional points plotted as open circles (“Orig.Tgt”) (in green) show the number of barcodes classified as cells for each targeted gene expression sample, analyzed using the standard cell-calling method (originally intended for non-targeted datasets) from Cell Ranger 3.1.

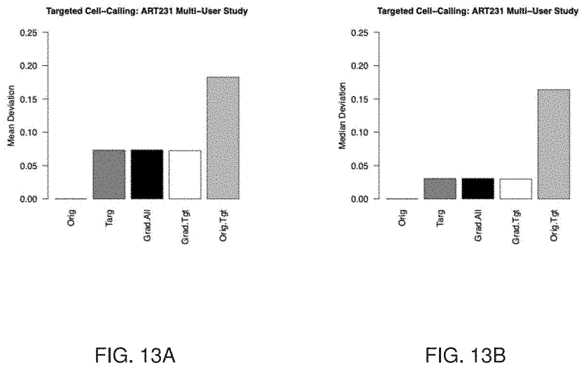

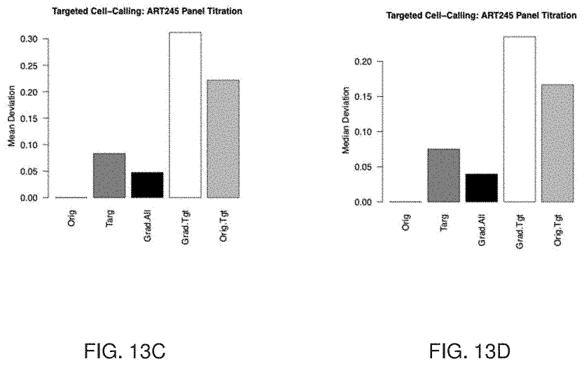

FIGS. 13A-D show bar graphs depicting the mean (left panels, 13A and 13C) and median (right panels, 13B and 13D) deviation between the number of barcodes called as cells across the set of targeted gene expression samples analyzed relative to the number of barcodes called as cells in each corresponding input WTA sample, in accordance with various embodiments. The deviation is computed as the absolute value of the difference between the number of cells called in a targeted gene expression sample and the number of cells called in its corresponding parent input WTA sample, divided by the number of cells called in the parent input WTA sample. Panels 13A-B depict results generated from the multi-user study presented in FIG. 10, while Panels 13C-D depict results generated from the multi-user study presented in FIG. 12.

Computer Implemented System

In various embodiments, the methods for identifying a subset of cells from a genomic sequence dataset for targeted gene expression analysis can be implemented via computer software or hardware. That is, as depicted in FIG. 6, the methods disclosed herein can be implemented on a computing device 606 that includes upstream processing engines 608, a unique molecule filtering engine 610 and downstream processing engines 612. In various embodiments, the computing device 606 can be communicatively connected to a data store 604 and a display device 614 via a direct connection or through an internet connection.

It should be appreciated that the various engines depicted in FIG. 6 can be combined or collapsed into a single engine, component or module, depending on the requirements of the particular application or system architecture. Moreover, in various embodiments, the upstream processing engines 608, unique molecule filtering engine 610 and downstream processing engines 612 can comprise additional engines or components as needed by the particular application or system architecture.

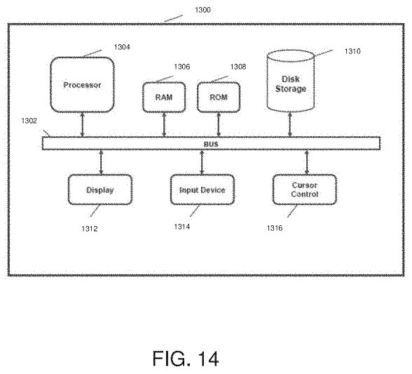

FIG. 14 is a block diagram illustrating a computer system 1400 upon which embodiments of the present teachings may be implemented. In various embodiments of the present teachings, computer system 1400 can include a bus 1402 or other communication mechanism for communicating information and a processor 1404 coupled with bus 1402 for processing information. In various embodiments, computer system 1400 can also include a memory, which can be a random-access memory (RAM) 1406 or other dynamic storage device, coupled to bus 1402 for determining instructions to be executed by processor 1404. Memory can also be used for storing temporary variables or other intermediate information during execution of instructions to be executed by processor 1404. In various embodiments, computer system 1400 can further include a read only memory (ROM) 1408 or other static storage device coupled to bus 1402 for storing static information and instructions for processor 1404. A storage device 1410, such as a magnetic disk or optical disk, can be provided and coupled to bus 1402 for storing information and instructions.

In various embodiments, computer system 1400 can be coupled via bus 1402 to a display 1412, such as a cathode ray tube (CRT) or liquid crystal display (LCD), for displaying information to a computer user. An input device 1414, including alphanumeric and other keys, can be coupled to bus 1402 for communication of information and command selections to processor 1404. Another type of user input device is a cursor control 1416, such as a mouse, a trackball or cursor direction keys for communicating direction information and command selections to processor 1404 and for controlling cursor movement on display 1412. This input device 1414 typically has two degrees of freedom in two axes, a first axis (i.e., x) and a second axis (i.e., y), that allows the device to specify positions in a plane. However, it should be understood that input devices 1414 allowing for 3-dimensional (x, y and z) cursor movement are also contemplated herein.

Consistent with certain implementations of the present teachings, results can be provided by computer system 1400 in response to processor 1404 executing one or more sequences of one or more instructions contained in memory 1406. Such instructions can be read into memory 1406 from another computer-readable medium or computer-readable storage medium, such as storage device 1410. Execution of the sequences of instructions contained in memory 1406 can cause processor 1404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1404 for execution. Such a medium can take many forms, including but not limited to, non-volatile media, volatile media, and transmission media. Examples of non-volatile media can include, but are not limited to, dynamic memory, such as memory 1406. Examples of transmission media can include, but are not limited to, coaxial cables, copper wire, and fiber optics, including the wires that comprise bus 1402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1404 of computer system 1400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 1300 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1300, whereby processor 1304 would execute the analyses and determinations provided by these engines, subject to instructions provided by any one of, or a combination of, memory components 1306/1308/1310 and user input provided via input device 1314.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### Recitation of Embodiments

Embodiment 1: A method for identifying a subset of cells from a dataset for targeted gene expression analysis, the method comprising defining an expected total recovered cell count for a barcode dataset, wherein the barcodes in the dataset are sorted based on count; determining a region of interest for the total recovered cell count by calculating a lower bound for the region of interest using a percentile of the sorted barcodes, wherein all barcodes having a molecule count above the lower bound are included for further analysis; calculating an upper bound for the region of interest by including an additional subset of barcodes having molecule counts below the lower bound, wherein each barcode in the subset that has a molecule count below a count threshold is excluded from further analysis; fitting the barcode dataset to a model, wherein the model groups barcodes as a function of molecule count; identifying a cut-off value on the model, wherein all barcodes having a molecule count above the cut-off value are called as cells; and identifying a targeted subset of cells from the called cells for conducting targeted gene expression, wherein a cell qualifies as part of the targeted subset if the cell has a molecule count above a target threshold.

Embodiment 2: The method of Embodiment 1, wherein the percentile is the 1^{st} percentile barcode of ranked barcodes.

Embodiment 3: The method of Embodiment 2, wherein the lower bound is 10 percent of the count of the 1^{st} percentile barcode.

Embodiment 4: The method of any one of Embodiments 1 to 3, wherein the additional subset of barcodes comprises about 2,000 barcodes.

Embodiment 5: The method of any one of Embodiments 1 to 4, wherein the count threshold is about 10 counts.

Embodiment 6: The method of any one of Embodiments 1 to 5, wherein the target threshold is a count greater than zero.

Embodiment 7: The method of any one of Embodiments 1 to 6, wherein the model is a plot of count as a function of ranked barcodes.

Embodiment 8: The method of Embodiment 7, wherein the plot is a log-transformed barcode-rank plot.

Embodiment 9: The method of any one of Embodiments 1 to 8, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

Embodiment 10: The method of any one of Embodiments 1 to 9, wherein the cut-off value is the point on the curve where the first derivative reaches a global minimum value.

Embodiment 11: A non-transitory computer-readable medium storing computer instructions for identifying a subset of cells from a dataset for targeted gene expression analysis, the method comprising defining, by one or more processors, an expected total recovered cell count for a barcode dataset, wherein the barcodes in the dataset are sorted based on count; determining, by one or more processors, a region of interest for the total recovered cell count by calculating a lower bound for the region of interest using a percentile of the sorted barcodes, wherein all barcodes having a molecule count above the lower bound are included for further analysis; calculating an upper bound for the region of interest by including an additional subset of barcodes below the lower bound, wherein each barcode in the subset that has a molecule count below a count threshold is excluded from further analysis; fitting, by one or more processors, the barcode dataset to a model, wherein the model groups barcodes as a function of molecule count; identifying, by one or more processors, a cut-off value on the model, wherein all barcodes having a molecule count above the cut-off value are called as cells; and identifying, by one or more processors, a targeted subset of cells from the called cells for conducting targeted gene expression, wherein a cell qualifies as part of the targeted subset if the cell has a molecule count above a target threshold.

Embodiment 12: The method of Embodiment 11, wherein the percentile is the 1^{st} percentile barcode of ranked barcodes.

Embodiment 13: The method of Embodiment 12, wherein the lower bound is 10 percent of the count of the 1^{st} percentile barcode.

Embodiment 14: The method of any one of Embodiments 11 to 13, wherein the additional subset of barcodes comprises about 2,000 barcodes.

Embodiment 15: The method of any one of Embodiments 11 to 14, wherein the count threshold is about 10 counts.

Embodiment 16: The method of any one of Embodiments 11 to 15, wherein the target threshold is a count greater than zero.

Embodiment 17: The method of any one of Embodiments 11 to 16, wherein the model is a plot of count as a function of ranked barcodes.

Embodiment 18: The method of Embodiment 17, wherein the plot is a log-transformed barcode-rank plot.

Embodiment 19: The method of any one of Embodiments 11 to 18, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

Embodiment 20: The method of any one of Embodiments 11 to 19, wherein the cut-off value is the point on the curve where the first derivative reaches a global minimum value.

Embodiment 20.5: The method of any one of Embodiments 1 to 20, wherein the barcode dataset comprises an antibody barcode dataset.

Embodiment 21: A system for identifying a subset of cells from a genomic sequence dataset for targeted gene expression analysis, comprising: a data store configured to store the genomic sequence dataset comprising a plurality of fragment sequence reads, each with an associated barcode sequence and a unique identifier sequence; and a computing device communicatively connected to the data store, comprising a unique molecule filtering engine configured to define an expected total recovered cell count for a barcode dataset, wherein the barcodes in the dataset are sorted based on count; determine a region of interest for the total recovered cell count by (a) calculating a lower bound for the region of interest using a percentile of the sorted barcodes, wherein all barcodes having a molecule count above the lower bound are included for further analysis; and (b) calculating an upper bound for the region of interest by including an additional subset of barcodes below the lower bound, wherein each barcode in the subset that has a molecule count below a count threshold is excluded from further analysis; fit the barcode dataset to a model, wherein the model groups barcodes as a function of molecule count; identify a cut-off value on the model, wherein all barcodes having a molecule count above the cut-off value are called as cells; and identify a targeted subset of cells from the called cells for conducting targeted gene expression, wherein a cell qualifies as part of the targeted subset if the cell has a molecule count above a target threshold.

Embodiment 22: The system of Embodiment 21, wherein the percentile is the 1^{st} percentile barcode of ranked barcodes.

Embodiment 23: The system of Embodiment 22, wherein the lower bound is 10 percent of the count of the 1^{st} percentile barcode.

Embodiment 24: The system of any one of Embodiments 21 to 23, wherein the additional subset of barcodes comprises about 2,000 barcodes.

Embodiment 25: The system of any one of Embodiments 21 to 24, wherein the count threshold is about 10 counts.

Embodiment 26: The system of any one of Embodiments 21 to 25, wherein the target threshold is a count greater than zero.

Embodiment 27: The system of any one of Embodiments 21 to 26, wherein the model is a plot of count as a function of ranked barcodes.

Embodiment 28: The system of Embodiment 27, wherein the plot is a log-transformed barcode-rank plot.

Embodiment 29: The system of any one of Embodiments 21 to 28, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

Embodiment 30: The system of any one of Embodiments 21 to 29, wherein the cut-off value is the point on the curve where the first derivative reaches a global minimum value.

Embodiment 31: The system of any one of Embodiments 21 to 30, further including: one or more upstream processing engines configured to process the genomic sequence data set prior to being received by the unique molecule filtering engine.

Embodiment 32: The system of any one of Embodiments 21 to 31, further including one or more downstream processing engines configured to process the identified targeted subset of cells generated by the unique molecule filtering engine.

Embodiment 33: The system of any one of Embodiments 21 to 32, wherein the data store and the computing device are part of an integrated apparatus.

Embodiment 34: The system of any one of Embodiments 21 to 33, wherein the data store is hosted by a different device than the computing device.

Embodiment 35: The system of any one of Embodiments 21 to 34, wherein the data store and the computing device are part of a distributed network system.

Embodiment 36: The system of any one of Embodiments 21 to 35, wherein the barcode dataset comprises an antibody barcode dataset.

1. A method for identifying a subset of cells from a dataset for targeted gene expression analysis, the method comprising:
defining an expected total recovered cell count for a barcode dataset, wherein the barcodes in the dataset are sorted based on count;
determining a region of interest for the total recovered cell count by
(a) calculating a lower bound for the region of interest using a percentile of the sorted barcodes, wherein all barcodes having a molecule count above the lower bound are included for further analysis;
(b) calculating an upper bound for the region of interest by including an additional subset of barcodes having molecule counts below the lower bound, wherein each barcode in the subset that has a molecule count below a count threshold is excluded from further analysis;
fitting the barcode dataset to a model, wherein the model groups barcodes as a function of molecule count;
identifying a cut-off value on the model, wherein all barcodes having a molecule count above the cut-off value are called as cells; and
identifying a targeted subset of cells from the called cells for conducting targeted gene expression, wherein a cell qualifies as part of the targeted subset if the cell has a molecule count above a target threshold.

2. The method of claim 1, wherein the percentile is the 1^{st} percentile barcode of ranked barcodes.

3. The method of claim 2, wherein the lower bound is 10 percent of the count of the 1^{st} percentile barcode.

4. The method of claim 1, wherein the additional subset of barcodes comprises about 2,000 barcodes.

5. The method of claim 1, wherein the count threshold is about 10 counts.

6. The method of claim 1, wherein the target threshold is a count greater than zero.

7. The method of claim 1, wherein the model is a plot of count as a function of ranked barcodes.

8. The method of claim 7, wherein the plot is a log-transformed barcode-rank plot.

9. The method of claim 1, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

10. The method of claim 1, wherein the cut-off value is the point on the curve where the first derivative reaches a global minimum value.

11. A non-transitory computer-readable medium storing computer instructions for identifying a subset of cells from a dataset for targeted gene expression analysis, the method comprising:
defining, by one or more processors, an expected total recovered cell count for a barcode dataset, wherein the barcodes in the dataset are sorted based on count;
determining, by one or more processors, a region of interest for the total recovered cell count by
(a) calculating a lower bound for the region of interest using a percentile of the sorted barcodes, wherein all barcodes having a molecule count above the lower bound are included for further analysis;
(b) calculating an upper bound for the region of interest by including an additional subset of barcodes below the lower bound, wherein each barcode in the subset that has a molecule count below a count threshold is excluded from further analysis;
fitting, by one or more processors, the barcode dataset to a model, wherein the model groups barcodes as a function of molecule count;
identifying, by one or more processors, a cut-off value on the model, wherein all barcodes having a molecule count above the cut-off value are called as cells; and
identifying, by one or more processors, a targeted subset of cells from the called cells for conducting targeted gene expression, wherein a cell qualifies as part of the targeted subset if the cell has a molecule count above a target threshold.

12. The method of claim 11, wherein the percentile is the 1^{st} percentile barcode of ranked barcodes.

13. The method of claim 12, wherein the lower bound is 10 percent of the count of the 1^{st} percentile barcode.

14. The method of claim 11, wherein the additional subset of barcodes comprises about 2,000 barcodes.

15. The method of claim 11, wherein the count threshold is about 10 counts.

16. The method of claim 11, wherein the target threshold is a count greater than zero.

17. The method of claim 11, wherein the model is a plot of count as a function of ranked barcodes.

18. The method of claim 17, wherein the plot is a log-transformed barcode-rank plot.

19. The method of claim 11, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

20. The method of claim 1, wherein the cut-off value is the point on the curve where the first derivative reaches a global minimum value.

21. A system for identifying a subset of cells from a genomic sequence dataset for targeted gene expression analysis, comprising:
a data store configured to store the genomic sequence dataset comprising a plurality of fragment sequence reads, each with an associated barcode sequence and a unique identifier sequence; and
a computing device communicatively connected to the data store, comprising a unique molecule filtering engine configured to:
define an expected total recovered cell count for a barcode dataset, wherein the barcodes in the dataset are sorted based on count;
determine a region of interest for the total recovered cell count by (a) calculating a lower bound for the region of interest using a percentile of the sorted barcodes, wherein all barcodes having a molecule count above the lower bound are included for further analysis; and (b) calculating an upper bound for the region of interest by including an additional subset of barcodes below the lower bound, wherein each barcode in the subset that has a molecule count below a count threshold is excluded from further analysis;
fit the barcode dataset to a model, wherein the model groups barcodes as a function of molecule count;
identify a cut-off value on the model, wherein all barcodes having a molecule count above the cut-off value are called as cells; and
identify a targeted subset of cells from the called cells for conducting targeted gene expression, wherein a cell qualifies as part of the targeted subset if the cell has a molecule count above a target threshold.

22. The system of claim 21, wherein the percentile is the 1^{st} percentile barcode of ranked barcodes.

23. The system of claim 22, wherein the lower bound is 10 percent of the count of the 1^{st} percentile barcode.

24. The system of claim 21, wherein the additional subset of barcodes comprises about 2,000 barcodes.

25. The system of claim 21, wherein the count threshold is about 10 counts.

26. The system of claim 21, wherein the target threshold is a count greater than zero.

27. The system of claim 21, wherein the model is a plot of count as a function of ranked barcodes.

28. The system of claim 27, wherein the plot is a log-transformed barcode-rank plot.

29. The system of claim 21, wherein the cut-off value on the model is determined by a mathematical method selected from the group consisting of a cubic spline curve fitted to a log-transformed barcode-rank plot, linear regression analysis, non-linear regression analysis, probability distribution fitting, smoothing, interpolation methods, trend estimation, least-squares estimation, function approximation, Loess fitting, and combinations thereof.

30. The system of claim 21, wherein the cut-off value is the point on the curve where the first derivative reaches a global minimum value.

31. The system of claim 21, further including:
one or more upstream processing engines configured to process the genomic sequence data set prior to being received by the unique molecule filtering engine.

32. The system of claim 21, further including:
one or more downstream processing engines configured to process the identified targeted subset of cells generated by the unique molecule filtering engine.

33. The system of claim 21, wherein the data store and the computing device are part of an integrated apparatus.

34. The system of claim 21, wherein the data store is hosted by a different device than the computing device.

35. The system of claim 21, wherein the data store and the computing device are part of a distributed network system.
